(19) United States
(12) Patent Application Publication
(10) Pub. No.: US 2007/0025951 A1
(43) Pub. Date: Feb. 1, 2007

(54) Skin care compositions containing xanthones

(76) Inventor: Sidney W. Foulger, Potomac, MD (US)

Correspondence Address: ALAN J. HOWARTH, P.O. BOX 1909, SANDY, UT, 84091-1909, US

(21) Appl. No.: 11/474,084

(22) Filed: Jun. 23, 2006

Related U.S. Application Data
(63) Continuation of application No. 11/127,798, filed on May 10, 2005, now abandoned.

(51) Int. Cl. A61K 8/97; A61K 31/353

(52) U.S. Cl. 424/74; 514/454

(57) ABSTRACT

Skin care compositions containing xanthones extracted from plants are described. These compositions include lotions, creams, ointments, and the like that reduce wrinkles, give skin a more youthful appearance, provide antioxidant effects, protect the skin, and moisturize the skin. The compositions contain an admixture of a base and a xanthone-containing plant extract.

### CROSS-REFERENCE TO RELATED APPLICATIONS

This application is a continuation of U.S. application Ser. No. 11/127,798, filed May 10, 2005, now abandoned, which is hereby incorporated in its entirety.

### STATEMENT REGARDING FEDERALLY SPONSORED RESEARCH OR DEVELOPMENT

Not applicable.

### BACKGROUND OF THE INVENTION

This invention relates to skin care compositions. More particularly, the invention relates to compositions for use in caring for the skin, wherein these compositions contain xanthones derived from plants. These compositions are useful for reducing wrinkles, making skin appear more youthful, providing antioxidative effects to the skin, and the like.

Plants have been used worldwide in traditional medicines for the treatment of diseases. It is estimated that even today approximately two-thirds to three-quarters of the world's population rely on medicinal plants as their primary source of medicines. In recent years, the physiological effects of foods (e.g., fruits, vegetables, nuts, and chocolate) and beverages (e.g., fruit juices, wine, tea, and coffee) rich in polyphenolic compounds have generated great interest in the scientific community. As dietary sources of biologically active compounds, these foods prove to be valuable for health. The antioxidant activity exhibited by these plant-derived phenolic compounds and their beneficial effects toward cardiovascular disorders, blood pressure, and high cholesterol have been the primary focus of attention of studies carried out over the past two decades on these compounds.

Oxidative damage to DNA, proteins, and other macromolecules resulting from accumulation of mutagens is considered to be one of the most important causes of degenerative diseases. Studies have shown that free radicals generated by oxidative and other biochemical pathways are important causes of this problem. Free radicals have been implicated in over 50 diseases. It has been estimated that at least 85% of chronic and degenerative diseases result from oxidative damage caused by free radicals. This suggests that free radicals are a fundamental component of tissue injury in many human diseases.

The human body has several mechanisms to counteract damage by free radicals and other reactive oxygen species. One important mechanism that guards against free radical damage is the action of antioxidants, which act as radical scavengers and convert the radicals to less reactive species. The imbalance due to the presence of excessive free radicals and low levels of antioxidants causes oxidative stress and is involved in many chronic health problems, such as cancer; atherosclerosis; myocardial infarction; arthritis; immune diseases such as multiple sclerosis, lupus, and scleroderma; neurodegenerative diseases such as Alzheimer's Disease and Parkinson's Disease; acquired immune deficiency syndrome (AIDS); cataracts; skin wrinkling; and generalized aging. To alleviate this imbalance, there is a general need to fortify various antioxidative mechanisms in the body.

Xanthones are naturally occurring antioxidants that are found in certain plants. Xanthones have found to be beneficial when added to the human diet. The present invention relates to additional benefits that xanthones provide when they are applied to the skin.

In view of the foregoing, it will be appreciated that providing skin care compositions comprising xanthone-containing extracts from xanthone-rich plant parts will provide greater amounts of such xanthones and corresponding greater benefits to users of such compositions.

### BRIEF SUMMARY OF THE INVENTION

It is a feature of the present invention to provide skin care compositions comprising extracts from xanthone-containing plants for providing beneficial effects to human skin, such as reducing wrinkles, making skin appear more youthful, providing antioxidative effects, protecting the skin, moisturizing the skin, and the like.

A skin care composition according to the present invention comprises an admixture of a base and a xanthone-containing plant extract. In one illustrative embodiment of the invention, the skin care composition comprises about 50 to about 99.999 parts by weight of the base and about 1×10⁻³ to about 50 parts by weight of the xanthone-containing plant extract. The base illustratively comprises an oleaginous base, such as a hydrocarbon base, a silicone polymer, a vegetable oil, or animal fat; an absorption base; an emulsion base, such as a base comprising an aqueous phase, an emulsifying agent, and an oleaginous phase; or a water-soluble base, such a base comprising polyethylene glycols, bentonite, colloidal magnesium aluminum silicate, sodium alginate, glyceryl monostearate, cellulose derivatives, or mixtures thereof. Cellulose derivatives include methylcellulose, hydroxyethyl cellulose, and sodium carboxymethyl cellulose, and mixtures thereof. The skin care compositions of the present invention can further comprise a member selected from the group consisting of proteins, vitamins, botanical extracts, lipids, glycolipids, polymers, copolymers, and the like, and mixtures thereof. Further, these compositions can further comprise a member selected from the group consisting of fragrances, coloring agents, emulsifiers, wetting agents, dispersing agents, penetrants, emollients, detergents, hardeners, preservatives, and the like, and mixtures thereof.

Another illustrative embodiment of the invention comprises a method for making a skin care composition, the method comprising admixing a base and a xanthone-containing plant extract.

Still another illustrative embodiment of the invention comprises a method for caring for skin, the method comprising applying to the skin a composition comprising an admixture of a base and a xanthone-containing plant extract.

### DETAILED DESCRIPTION

Before the present skin care compositions containing xanthones are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to a skin care composition comprising “a xanthone” includes reference to two or more of such xanthones, reference to “an emulsifier” includes reference to two or more of such emulsifiers, and reference to “the preservative” includes reference to two or more of such preservatives.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.” As used herein, “consisting of” and grammatical equivalents thereof exclude any element, step, or ingredient not specified in the claim. As used herein, “consisting essentially of” and grammatical equivalents thereof limit the scope of a claim to the specified materials or steps and those that do not materially affect the basic and novel characteristic or characteristics of the claimed invention.

As used herein, “lotions” are liquid cosmetics, often suspensions or dispersions intended for external application to the body.

As used herein, “creams” are soft cosmetic-type preparations. Creams of the oil-in-water (O/W) type include preparations such as foundation creams, hand creams, shaving creams, and the like. Creams of the water-in-oil (W/O) type include cold creams, emollient creams, and the like. Pharmaceutically, creams are solid emulsions containing suspensions or solutions of active ingredients for external application. Generally, preparations of this type are classified as ointments. Specifically, they belong to the emulsion-type bases.

As used herein, “ointments” are semisolid preparations for external application of such consistency that may be readily applied to the skin. They should be of such composition that they soften, but not necessarily melt, when applied to the body. They serve as vehicles for the topical application of active ingredients and also function as protectives and emollients for the skin. For many years ointments were limited by definition and use to mixtures of fatty substances. Today, in addition to such oleaginous mixtures, there are ointment preparations possessing the same general consistency but entirely free of oleaginous substances. In many instances, they are emulsions of fatty or wax-like materials with comparatively high proportions of water. These emulsions may be either water-in-oil (W/O) or oil-in-water (O/W) emulsions, depending primarily on the selection of the emulsifying agent. Such semisolid emulsions are also referred to as creams. Creams and ointments containing large amounts of insoluble powders are referred to as pastes. Pastes are usually stiffer and more absorptive than creams and ointments.

Xanthones

This invention relates to skin care compositions containing xanthone compounds extracted from xanthone-rich medicinal plants, such as Garcinia mangostana (known as the mangosteen plant), Kielmeyera variabilis, and Swertia davidi, and the like. These xanthones are extracted from the plant sources using solvents, and then are combined with a base to result in a skin care composition.

Mangosteen is a tree that is fairly widespread in Southeast Asia and is known for its medicinal properties. The fruit hulls have been used in folk medicine for the treatment of skin infections, wounds, and diarrhea in Southeast Asia. Kielmeyera variabilis is a tree commonly known in Brazil as “malva-do-camp” or “pausanto.” It has been used in traditional Brazilian folk medicine for treatment of several tropical diseases, including schistosomiasis, leishmaniasis, malaria, and fungal and bacterial infections. Swertia davidi is a Chinese herb that has been used in treatment of inflammation, allergy, and hepatitis.

Phytochemical studies have shown that these and other plant species are rich in a variety of xanthones, which have demonstrated a number of bioactivities and pharmacological activities. In the last decade, more than 700 scientific papers have been published on the structures, bioactivities, and pharmacological activities of xanthones, and more than 100 different effects have been noted. The bioactivities and pharmacological activities of xanthones include, but are not limited to: antioxidant, antifungal and antibacterial, cardiovascular protective, antitumor and cancer protective, anti-aging, anti-human immunodeficiency virus (anti-HIV), antilipidemic (blood-fat lowering), hypotensive (lowering blood pressure), stimulate immune responses, antidiabetic effects (hypoglycemic; lower blood sugar level), antiobesity, antifatigue, antiathersclerotic (prevents hardening of arteries), antiviral, antidepressant, anti-anxiety, anti-Alzheimererian and anti-Parkinsonism (and other neurodegenerative diseases), antipyretic (lowering fever), antiperiodontic (prevents gum disease), antiallergenic (prevents allergic reactions), antiseborrheic, antiosteoporosis (prevents loss of bone mass), anticalculitic (prevents kidney stones), antidiarrheal, antineuralgic (reduces nerve pain), antiarthritic (prevents arthritis), anticataract (prevents cataracts), antiglaucomic (prevents glaucoma), anti-inflammatory, and anti-ulcer (prevents stomach, mouth, and bowel ulcers) agents.

Xanthones are water insoluble and thus are more readily extracted from plant tissues using organic solvents than aqueous solvents. For example, Nilar & L. J. Harrison, Xanthones from the heartwood of Garcinia mangostana, 60 Phytochemistry 541-548 (2002), describes extraction of xanthones from mangosteen heartwood using hot hexane. The hexane extract was concentrated to yield a crude extract, which was subjected to column chromatography on a silica column using an ethyl acetate-hexane gradient. The resulting fractions were subjected to gel permeation chromatography and high pressure liquid chromatograph (HPLC), which resulted in isolation of numerous xanthones. A. Groweiss et al., HIV-Inhibitory prenylated xanthones and flavones from Maclura tinctoria, 63 J. Nat. Prod. 1537-1539 (2000), describes extraction of xanthones from bark using ethylene chloride-methanol (1:1) as a solvent. C. Gopalakrishnan et al., Anti-inflammatory and C.N.S. depressant activities of xanthones from Calophyllum inophyllum and Mesua ferrea, 12 Ind. J. Pharmac. 181-191 (1980), found that xanthones were freely soluble in n-hexane, benzene, ethanol, and chloroform. C. Ito et al., Cancer chemopreventive agents. New depsidones from Garcinia plants, 64 J. Nat. Prod. 147-150 (2001), described extraction of xanthones from dried leaves using acetone. L. Pinheiro et al., Antibacterial xanthones from Kielmeyera variabilis Mart. (Clusiaceae), 98 Mem. Inst. Oswaldo Cruz 549-552 (2003), describes extraction of xanthones from malva-do-campo with hexane and methanol. D. J. Jiang et al., Demethylbellidifolin preserves endothelial function by reduction of the endogenous nitric oxide synthase inhibitor level, 93 J. Ethnopharmacology 295-306 (2004), described extraction of xanthones from dried plants using ethanol. Thus, various organic solvents can be used to effectively extract xanthones from plant material.

Illustratively, according to the present invention xanthones were extracted from plants by extraction in hexane-methanol or ethanol. The resulting organic extract was then treated by rotary evaporation to evaporate the solvent, thus resulting in crude xanthones as a water-insoluble gum plus a water-soluble fraction. The water-insoluble gum was then lyophilized, resulting in a xanthone powder.

Bases for Skin Care Compositions

Ideally, an ointment base should be nonirritating, nondehydrating, nongreasy, compatible with active ingredients, stable, easily removable with water, absorptive (able to absorb water and/or other liquids), and able to efficiently release the incorporated active ingredients. No ointment base possesses all of these characteristics. Ointments can be classified according to their composition. Ointment classes include oleaginous bases, absorption bases, emulsion bases, and water-soluble bases.

Oleaginous bases are generally anhydrous, hydrophobic, insoluble in water, and are not water-removable. Oleaginous bases includes the early ointments, which consisted almost entirely of vegetable and animal fats, as well as petroleum hydrocarbons. Fixed oils of vegetable origin include olive, cottonseed, sesame, persic, and other oils. Hydrocarbon bases include ointments prepared from petrolatum or liquid petrolatum with wax or other stiffening agents. Hydrocarbon bases do not become rancid, which is an advantage compared to animal fats and vegetable oils. Another oleaginous base includes silicones, which are synthetic polymers in which the basic structure is an alternating chain of silicon and oxygen atoms (e.g., —O—Si—O—Si—O—Si—). Silicones used in the pharmaceutical and cosmetic industries include dimethylpolysiloxane, methylphenylpolysiloxane, and a stearyl ester of dimethylpolysiloxane, all of which are insoluble in water and are water repellant. Illustrative oleaginous bases are well known in the art, such as Silicone Gibson Base (Example 1) and Vanisil Silicone Ointment (Example 2).

Absorption bases are generally anhydrous, hydrophilic, insoluble in water, and most are not water-removable. These bases have the property of absorbing several times their weight of water and forming emulsions while retaining their ointment-like consistency. Absorption bases vary in their composition, but for the greater part, they are mixtures of animal sterols with petrolatum. Combinations of cholesterol and/or other lanolin fractions with white petrolatum are such absorption bases, and Eucerin and Aquaphor were among the earliest commercial bases of this type. Zopf Emollient Cream (Example 3), Hoch Formula Base (Example 4), Hydrophilic Petrolatum Base (Example 5), Wool Alcohols Ointment Base (Example 6), and Aquabase Ointment Base (Example 7) are absorption bases described herein. Some commercially available absorption bases include Aquafor, Polysorb (Fougera), and Nivea Cream.

Emulsion bases can be either W/O bases, which are hydrous, insoluble in water, and not removable with water and will absorb water, or O/W bases, which are hydrous, insoluble in water, and water removable and will absorb water. These preparations are solid emulsions, and similar products have long been used as cosmetic creams. The availability of numerous compounds for use as wetting agents, dispersing agents, emulsifiers, penetrants, emollients, detergents, hardeners, preservatives, and the like has given a great deal of flexibility to ointment formulation. Although surface-active agents (i.e., surfactants) may be ionic or nonionic, the nonionic agents are widely used in dermatologic and pharmaceutical preparations. Polysorbate 80 (e.g., Tween 80) and Polyoxyl 40 Stearate represent such surfactants. Nonionic surfactants are generally less toxic and less irritating than ionic surfactants. Other advantages include their virtual neutrality, stability to freezing, stability to electrolytes, and ease of use. In general, the emulsion bases contain an aqueous phase, an emulsifying agent, and an oleaginous phase. The water phase of illustrative emulsion bases typically varies from 10 to 80% by weight of the total base. Glycerin, propylene glycol, or a polyethylene glycol is generally included with the aqueous phase to serve as a humectant, to reduce water loss through evaporation, and to lend a general softness to the creams. The addition of certain alcohols to emulsion base formulas also adds stability to the emulsion and imparts a smooth feel to the skin. Stearyl alcohol, a solid, increases the consistency of the ointment and permits the incorporation of more liquid components. Due to their ability to become hydrated, such alcohols assist in water retention of emulsion bases. The oleaginous phase may contain one or more of the following or similar ingredients: petrolatum, fats, waxes, organic alcohols, polyglycol esters, or other grease-like substances. These substances are emulsified with the aqueous phase through the action of the surfactant. A few such emulsifiers include alkali soaps, alkyl sulfates, amine soaps, polyglycol esters, alkyl aryl sulfates, quaternary ammonium compounds, and the like. These emulsifying compounds aid in the dispersion of the fats and waxes in water and increase the stability of the ointments. Hydrophilic Ointment Base (Example 10), Beeler's Base (Example 11), and U.C.H. Base (Example 12) are illustrative O/W emulsion bases described herein. Commercially available O/W emulsion bases include Cetaphil Cream, Neobase, Unibase, Dermovan, Phorsix Cream, Lubriderm Cream, and Velvachol.

Water-soluble bases are anhydrous, soluble in water, water removable, and greaseless, and will absorb water. These bases include those bases prepared from polyethylene glycols as well as semisolid preparations containing bentonite, colloidal magnesium aluminum silicate, and sodium alginate. Polyethylene glycol (PEG) compounds 1500, 1540, 4000, and 6000 are of interest in ointment and lotion formulations. PEG 1500 is a soft waxy solid, similar in consistency to petrolatum, with a congealing range of 40° C. to 45° C. PEG 1540 is a solid of consistency of beeswax and is intermediate in physical properties between the 1500 and 4000 PEGs. PEG 4000 has a congealing range of 53° C. to 56° C. and is most useful as a component of being an ointment base for, in addition to the general property of being an emulsifying and dispersing agent, it also adds to the consistency of the base. Both PEG 4000 and PEG 6000 are nonhygroscopic. PEG 6000 is a hard, translucent, waxy solid, and has a congealing range of 58° C. to 62° C.

Glyceryl monostearate base is a polyhydric alcohol ester that has been widely used in cosmetic and ointment bases. It has a high melting point (56° C. to 58° C.) and is a good emulsifying agent. Glyceryl monostearate emulsions generally contain high water phases, usually above 60% by weight. It has the disadvantage of being incompatible with acids. Glyceryl monostearate base is described in Example 22.

Cellulose derivatives, such as methylcellulose and hydroxyethyl cellulose, form colloidal solutions that resemble gums and mucilages, but are not as vulnerable to fungal or bacterial attack. Methylcellulose is dispersible in cold water, but in concentrated solutions will coagulate upon heating. Hydroxyethyl cellulose is more soluble at elevated temperatures so that viscosity of aqueous solutions decreases slightly on warming. It is a good protective colloid for aqueous dispersions of oils, waxes, and pigments. Sodium carboxymethylcellulose is another cellulose derivative frequently referred to as carboxymethyl cellulose or CMC. It is an anionic compound and thereby can be used as a thickening or stabilizing agent for suspensions and for ointments of the emulsion type where the emulsifying agent is anionic or nonionic. Any of these cellulose derivatives can be used to stabilize ointment formulas, and they are commercially available in various viscosity types and with various degrees of substitution.

Sodium alginate is a hydrophilic colloid that is compatible with small amounts of alcohol, glycerin, polyglycols, wetting agents, and solutions of alkali carbonates. It functions satisfactorily under acid or alkaline conditions within the pH range of 4.5-10. It is possible to make sodium alginate solutions into semi-firm or firm gels by the addition of small amounts of soluble calcium salts, i.e., calcium gluconate, calcium tartrate, and calcium citrate. Ions of the alkaline earth metals will thicken or gelatinize sodium alginate solutions when present in low concentrations, while at high concentrations they will precipitate them. A 2.5% solution of sodium alginate is a satisfactory inert diluent for greaseless and other types of ointments.

Bentonite, a colloidal hydrated aluminum silicate, is insoluble in water, but when mixed with 8 to 10 parts of water it swells to produce a slightly alkaline gel resembling petrolatum. The consistency of the product may be regulated by varying the amounts of water added. Ointments prepared from bentonite and water alone are found to be slightly drying and unstable upon standing, but addition of a humectant, such as glycerin or sorbitol, in amounts up to about 10% by weight will retard this action. Ointments prepared from bentonite do not encourage mold growth, and they have the advantage of not spreading to the hair when applied to the scalp.

Colloidal magnesium aluminum silicate (e.g., Veegum®, R.T. Vanderbilt Company, Inc.) is an inorganic emulsifier, suspending agent, and thickener. Dispersions are slightly alkaline and are compatible with about 20 to 30% ethyl alcohol, isopropyl alcohol, acetone, and similar solvents. Glycols, such as glycerin and propylene glycol, are compatible at 40 to 50% concentrations.

Carbopol 934 (carboxypolymethylene) is an acid polymer that disperses readily in water to yield an acid solution of low viscosity. When the acid solution is neutralized with a suitable base, such as sodium bicarbonate, sodium hydroxide, or the like, a clear, stable gel results. Carbopol 934 is inert physiologically and is neither a primary irritant nor a sensitizer. The thickening efficiency of Carbopol 934 can be used in the preparation of such pharmaceuticals as creams, ointments, lotions, suspensions, and emulsions.

The skin care compositions of the present invention can also contain fragrances, proteins, colorants or coloring agents, lipids, vitamins, botanical extracts, lipids, glycolipids, polymers, and copolymers, and the like, as are generally known in the art of making skin care products. The Cosmetic, Toiletry, and Fragrance Association's International Cosmetic Ingredient Dictionary and Handbook is an excellent source of information concerning such ingredients.

As used herein, “colorants” or “coloring agents” are agents that give skin care compositions a more pleasing appearance, and in addition help the manufacturer to control the product during its preparation and help the user to identify the product. Any of the approved certified water-soluble FD&C dyes, mixtures thereof, or their corresponding lakes may be used to color skin care compositions. A color lake is the combination by adsorption of a water-soluble dye to a hydrous oxide of a heavy metal, resulting in an insoluble form of the dye.

The skin care compositions of the present invention are made by mixing a selected amount of the xanthone-containing extract with a selected skin care base and any other selected ingredients, such as coloring agents, fragrances, emollients, and the like.

The skin care compositions of the present invention are applied to the skin in amounts selected by the user. The compositions are dispensed from appropriate containers and are generally manually applied to the skin, as is well known in the art.

### EXAMPLE 1

Silicone Gibson Base

The following formula illustrates a silicone base that can be used in a cream or lotion according to the present invention. Silicone Gibson base comprises 15 parts by weight of cetyl alcohol, 1 parts by weight of sodium lauryl sulfate, 40 parts by weight of dimethylpolysiloxane polymer (1000 cps), 43 parts by weight purified water, 0.25 parts by weight methylparaben, and 0.15 parts by weight propylparaben. The aqueous mixture of the sodium lauryl sulfate and the parabens is warmed to 75° C., and then it is slowly added to warmed (25° C.) cetyl alcohol-silicone mixture. The resulting mixture is stirred until it congeals.

### EXAMPLE 2

Vanisil Silicone Ointment Base

The following formula illustrates a silicone base that can be used in a cream or lotion according to the present invention. Vanisil silicone ointment base comprises 10 parts by weight stearic acid, 2 parts by weight synthetic Japan wax, 20 parts by weight dimethylpolysiloxane polymer (1000 cps), 0.5 parts by weight potassium hydroxide, 0.025 parts by weight methylparaben, 0.015 parts by weight propylparaben, and 67.5 parts by weight distilled water.

### EXAMPLE 3

Zopf Emollient Cream

The following formula illustrates a W/O emulsion absorption base that can be used according to the present invention. Zopf emollient cream comprises 41 parts by weight of white petrolatum, 3 parts by weight of microcrystalline wax, 10 parts by weight of fluid lanolin, 4.75 parts by weight sorbitan monooleate, 0.25 parts by weight of polysorbate 80, and 41 parts by weight purified water. The aqueous dispersion of sorbitan monooleate and polysorbate 80 is warmed to 75° C. and then slowly added to the melted wax, white petrolatum, and fluid lanolin. The resulting mixture is stirred until it congeals.

### EXAMPLE 4

Hoch Formula Base

The following formula illustrates an O/W emulsion absorption base that can be used according to the present invention. Hoch formula base comprises phase A comprising 5 parts by weight of fluid lanolin, 35 parts by weight of castor oil, 2 parts by weight of sorbitan monostearate, 36.7 parts by weight of mineral oil, 4 parts by weight of stearic acid, and 0.2 parts by weight of propylparaben; and phase B comprising 1 parts by weight of polyethylene 20 sorbitan monostearate, 0.9 parts by weight of triethanolamine, 0.2 parts by weight of methylparaben, and 15 parts by weight of purified water. Phase A is heated to 78° C., and phase B is heated to 70° C. Then, phase B is added to phase A and the resulting mixture is stirred until it cools to 25° C.

### EXAMPLE 5

Hydrophilic Petrolatum Base

The following formula illustrates an absorption base that can be used according to the present invention. Hydrophilic petrolatum base comprises 30 parts by weight of cholesterol, 30 parts by weight of stearyl alcohol, 80 parts by weight of white wax, and 860 parts by weight of white petrolatum. The stearyl alcohol, white wax, and white petrolatum are melted together on a steam bath, and then the cholesterol is added and stirred into the mixture until the cholesterol completely dissolves. The mixture is then removed from the bath and stirred until it congeals.

### EXAMPLE 6

Wool Alcohols Base

The following formula illustrates an absorption base that can be used according to the present invention. Wool alcohols ointment base comprises 60 parts by weight wool alcohol, 240 parts by weight hard paraffin, 100 parts by weight white or yellow soft paraffin, and 600 parts by weight liquid paraffin. The ingredients are mixed together and stirred until cold.

### EXAMPLE 7

Aquabase Ointment Base

The following formula illustrates an absorption base that can be used according to the present invention. Aquabase ointment base comprises 30 parts by weight of cholesterol, 30 parts by weight of cottonseed oil, and 940 parts by weight of white petrolatum. The white petrolatum and cottonseed oil are heated to 145° C. and then removed from the heat. The cholesterol is then added and stirred until it is almost congealed. Then the ointment is placed in suitable containers.

### EXAMPLE 8

Emulsion Base

The following formula illustrates an emulsion base that can be used according to the present invention. Many dermatologic and cosmetic preparations contain amine soaps as emulsifying agents. These anionic emulsifiers are advantageous as compared to sodium and potassium soaps because they yield emulsions having a relatively low pH of about 8.0. Triethanolamine is generally used, along with a fatty acid, to produce the fatty acid amine soap. Triethanolamine usually contains small amounts of ethanolamine and diethanolamine. It combines stoichiometrically with fatty acids. Semisolid O/W bases containing triethanolamine soaps are generally prepared by dissolving the triethanolamine in water and then adding this solution to the oil phase with stirring. A typical formula for such a base comprises 18 parts by weight stearic acid, 4 parts by weight of cetyl alcohol, 2 parts by weight of triethanolamine, 5 parts by weight of glycerin, and 71 parts by weight of distilled water.

### EXAMPLE 9

Coal Tar Ointment Base

The following formula illustrates an emulsion base that can be used according to the present invention. Coal tar ointment base contains a surfactant, i.e., polysorbate 80, which serves the dual purpose of a dispersing agent and aiding in removal of the ointment from the skin. Coal tar ointment base comprises 10 parts by weight coal tar, 5 parts by weight polysorbate 80, and 985 parts by weight zinc oxide paste. The coal tar is blended with the polysorbate 80, and this blend is then mixed with the zinc oxide paste.

### EXAMPLE 10

Hydrophilic Ointment Base

The following formula illustrates an emulsion base that can be used according to the present invention. Hydrophilic ointment base comprises 0.25 parts by weight methylparaben, 0.15 parts by weight propylparaben, 10 parts by weight sodium lauryl sulfate, 120 parts by weight propylene glycol, 250 parts by weight stearyl alcohol, 250 parts by weight white petrolatum, and 370 parts by weight water. The stearyl alcohol and white petrolatum are melted on a steam bath and warmed to about 75° C. The other ingredients, previously dissolved in the water, are warmed to 75° C. and then added with stirring until the mixture congeals.

### EXAMPLE 11

Beeler's Base

The following formula illustrates an O/W emulsion base that can be used according to the present invention. Beeler's base comprises 15 parts by weight cetyl alcohol, 1 parts by weight white wax, 10 parts by weight propylene glycol, 2 parts by weight sodium lauryl sulfate, and 72 parts by weight water. The cetyl alcohol and white wax are melted in the propylene glycol on a water bath, and the resulting mixture is heated to about 65° C. The sodium lauryl sulfate is dissolved in the water and also heated on water bath to about 65° C. The oil phase is slowly added to the well-stirred water phase, and stirring is continued on the water bath for about 10 min. The emulsion is then removed from the water bath and stirring is continued to the point of congealing.

### EXAMPLE 12

U.C.H. Base

The following formula illustrates an emulsion base that can be used according to the present invention. U.C.H. base comprises 6.4 parts by weight cetyl alcohol, 5.4 parts by weight stearyl alcohol, 1.5 parts by weight sodium lauryl sulfate, 14.3 parts by weight white petrolatum, 21.4 parts by weight mineral oil, and 50 parts by weight water. The alcohols are melted together over a water bath at 65° C., then the sodium lauryl sulfate is add with stirring. Next the white petrolatum and the mineral oil are added with continued heating of the mixture until it is completely melted. This mixture is then cooled to room temperature and the water is added with constant mixing to result in the emulsion.

### EXAMPLE 13

Base A

The following formula illustrates an anhydrous emulsifiable solid mixture. Anhydrous solid mixture A is made by melting together 53 parts by weight of stearyl alcohol, 7 parts by weight of cetyl alcohol, 38.6 parts by weight of PEG 400, and 1.4 parts by weight of sodium lauryl sulfate. These ingredients are melted and stirred vigorously until completely solidified. Stirring is continued to insure complete mixing of the ingredients and for the production of a granular product. Base A is made by melting 50 parts by weight of the granular solid mixture A, heating it to 70-75° C., and then adding it to 50 parts by weight of an aqueous mixture at the same temperature. The mixture is stirred until the emulsion begins to solidify and cools to 40° C. The resulting base is a white, semisolid O/W emulsion of ointment-like consistency. It is non-greasy and washable with water. The emulsion is stable up to 55-60° C., exhibits a good sheen, and exhibits good lubricity when applied to skin.

### EXAMPLE 14

Base B

The following formula illustrates an anhydrous emulsifiable solid mixture. Anhydrous solid mixture B is made by melting together 64.7 parts by weight of stearyl alcohol, 8.6 parts by weight of cetyl alcohol, 13 parts by weight of PEG 1000 monostearate, 8.7 parts by weight of PEG 1540, and 5 parts by weight of anhydrous lanolin. These ingredients are melted and stirred vigorously until completely solidified. Stirring is continued to insure complete mixing of the ingredients and for the production of a granular product. Base B is made by melting 40 parts by weight of the granular solid mixture B, heating it to 70-75° C., and then adding it to 60 parts by weight of an aqueous mixture at the same temperature. The mixture is stirred until the emulsion begins to solidify and cools to 40° C. The resulting base is a white, semisolid O/W emulsion of ointment-like consistency. It is non-greasy and washable with water. The emulsion is stable up to 55-60° C. and exhibits good lubricity when applied to skin.

### EXAMPLE 15

Aqueous Cream Base

The following formula illustrates an emulsion base that can be used according to the present invention. Aqueous cream base is an emulsion base prepared from 30% by weight of emulsifying ointment and 70% by weight of water. Emulsifying ointment comprises 30 parts by weight emulsifying wax, 20 parts by weight liquid paraffin, and 50 parts by weight white soft paraffin. Emulsifying wax comprises 90 parts by weight cetostearyl alcohol, 10 parts by weight sodium lauryl sulfate, and 4 parts by weight purified water.

### EXAMPLE 16

Polyethylene Glycol Ointment Base

The following formula illustrates a water-soluble base that can be used according to the present invention. Polyethylene glycol ointment base comprises 400 parts by weight of PEG 4000 and 600 parts by weight of PEG 400. The two ingredients are heated on a water bath to 65° C., and then the mixture is allowed to cool with stirring until it congeals. If a firmer preparation is desired, up to 100 parts by weight of the PEG 400 can be replaced with an equal amount of PEG 4000. If 6-25% by weight of an aqueous solution is to incorporated in this polyethylene ointment, 50 parts by weight of the PEG 4000 is replaced with an equal amount of stearyl alcohol.

### EXAMPLE 17

Base G

The following formula illustrates a water-soluble base that can be used according to the present invention. The addition of an ester of polyethylene glycol to a polyethylene glycol ointment yields a water-removable, emulsifiable ointment base. An illustrative emulsifiable glycol ointment base (Base G) of this type comprises 26 parts by weight polyethylene glycol 400 monostearate, 37 parts by weight PEG 400, and 37 parts by weight PEG 4000. The glycols are mixed and melted at about 65° C. This mixture is then stirred while cooling to about 40° C. The polyethylene glycol 400 monostearate is melted at about 40° C. and then added to the liquid glycol mixture with stirring until a uniform ointment is obtained. Water (10-15% by weight) can be incorporated into Base G.

### EXAMPLE 18

Base III

The following formula illustrates a water-soluble base that can be used according to the present invention. Surfactants and water can be added to a polyethylene glycol ointment without impairing the water removability of the base. Base III represents a typical formula of this type: 50 parts by weight PEG 4000, 40 parts by weight PEG 400, 1 parts by weight sorbitan monopalmitate, and 9 parts by weight water. The sorbitan monopalmitate and the polyethylene glycols are warmed together on a water bath to 70° C. and the water heated to the same temperature is then added. The emulsion is stirred until it congeals.

### EXAMPLE 19

Modified Landon-Zopf Base

The following formula illustrates a water-soluble base that can be used according to the present invention. Modified Landon-Zopf base comprises 20 parts by weight PEG 4000, 34 parts by weight stearyl alcohol, 30 parts by weight glycerin, 15 parts by weight water, and 1 parts by weight sodium lauryl sulfate. The PEG 4000, stearyl alcohol, and glycerin are heated on a water bath to 75° C. This mixture is then added in small quantities with stirring to the water, which contains the sodium lauryl sulfate and has also been heated to 75° C. Moderate stirring is continued until the base has congealed.

### EXAMPLE 20

Canadian Base

The following formula illustrates a water-soluble base that can be used according to the present invention. Canadian base comprises 11.2 parts by weight PEG 4000, 20.8 parts by weight stearyl alcohol, 17 parts by weight glycerin, 0.6 parts by weight sodium lauryl sulfate, and 50.4 parts by weight water. The PEG 4000, stearyl alcohol, and glycerin are heated on a water bath to 70° C. The water, which contains the sodium lauryl sulfate and has been previously heated to 70° C., is added and the mixture is stirred until the base congeals.

### EXAMPLE 21

Base IV

The following formula illustrates a water-soluble base that can be used according to the present invention. Base IV comprises 42.5 parts by weight PEG 4000, 37.5 parts by weight PEG 400, and 20 parts by weight 1,2,6-hexanetriol. The PEG 4000 is heated with the 1,2,6-hexanetriol is heated on a water bath to 60-70° C. This mixture is added to the PEG 400 at room temperature with vigorous stirring. The, occasional stirring is continued until solidification takes place.

### EXAMPLE 22

Glyceryl Monostearate Base

The following formula illustrates a water-soluble base that can be used according to the present invention. Glyceryl monostearate base comprises 10 parts by weight mineral oil, 30 parts by weight white petrolatum, 10 parts by weight glyceryl monostearate S. E., 5 parts by weight cetyl alcohol, 5 parts by weight glycerin, and 40 parts by weight water.

### EXAMPLE 23

Lubricating Jelly Base

The following formula illustrates a water-soluble base that can be used according to the present invention. Lubricating jelly base comprises 1 g methocel 90 HC 4000, 0.3 g carbopol 934, sodium hydroxide qs pH 7.0, 20 ml propylene glycol, 0.15 g methylparaben, and purified water qs 100 g. The methocel is added slowly to 40 ml of hot water (80-90° C.) and agitated for 5 min. After cooling, the solution is refrigerated overnight. The carbopol 934 is dissolved in 20 ml of water, and 1% sodium hydroxide is added slowly with cautious stirring to avoid incorporation of air, until a pH of 7.0 is obtained, and then water is added to a total volume of 40 ml. The methylparaben is dissolved in the propylene glycol. Finally the methocel, carbopol, and methylparaben solutions are mixed cautiously to avoid incorporation of air.

### EXAMPLE 24

Universal O/W Ointment Base

The following formula illustrates a water-soluble base that can be used according to the present invention. Universal O/W ointment base comprises 0.05 parts by weight calcium citrate, 3 parts by weight sodium alginate, 0.20 parts by weight methylparaben, 45 parts by weight glycerin, and sufficient distilled water to make a total of 100 parts by weight. The calcium citrate and the methylparaben are dissolved in the water. The glycerin is mixed with the sodium alginate to form a smooth paste. The aqueous mixture is added to the paste and is stirred until a smooth, stiff preparation is obtained. The base is then set aside for several hours until thickening is complete.

### EXAMPLE 25

Hollander and McClanahan Base

The following formula illustrates a water-soluble base that can be used according to the present invention. Hollander and McClanahan base comprises 32 parts by weight petrolatum, 13 parts by weight bentonite, 0.5 parts by weight sodium lauryl sulfate, 54 parts by weight water, and 0.1 parts by weight methylparaben.

### EXAMPLE 26

MGH Ointment Base

The following formula illustrates a water-soluble base that can be used according to the present invention. MGH ointment base comprises 15 parts by weight polyethylene glycol 200 monostearate, 2.5 parts by weight colloidal magnesium stearate silicate (Veegum), 1 parts by weight polysorbate 80, 0.1 parts by weight methylparaben, and 81.4 parts by weight purified water.

### EXAMPLE 27

Lotion Base

The following formula illustrates a water-soluble base that can be used according to the present invention. Lotion base comprises 1 parts by weight Veegum, 0.85 parts by weight sodium carboxymethylcellulose, 90.15 parts by weight water, 3 parts by weight glycerin, and 5 parts by weight dioctyl sodium sulfosuccinate (1% solution). All the dry ingredients are mixed with water and glycerin in a blender for 1 min. The mixture is then removed from the blender and the dioctyl sodium sulfosuccinate is added.

### EXAMPLE 28

Cold Cream Base

The following formula illustrates a cold cream according to the present invention. A cold cream base comprises 6 parts by weight spermaceti, 6 parts by weight beeswax, 10 parts by weight Carbopol 934, 4.75 parts by weight sodium carbonate, 5 parts by weight rose water, 0.02 parts by weight rose oil, 56 parts by weight expressed almond oil, and 20 parts by weight distilled water.

### EXAMPLE 29

Hand Lotion Base

The following formula illustrates a hand lotion according to the present invention. A hand lotion base comprises 24.75 ml propylene glycol, 1 ml triethanolamine, 12 ml water, 1.5 g oleic acid, 10.5 g polyethylene glycol 400 monostearate, 10 ml silicone fluid D.C. 200, and 50 g carbopol 934 2% mucilage.

### EXAMPLE 30

White Lotion Base

White lotion base comprises 40 parts by weight zinc sulfate, 40 parts by weight sulfurated potash, and sufficient purified water to make 1000 parts by weight. The zinc sulfate and the sulfurated potash are dissolved separately, each in 450 parts by weight of purified water, and then each solution is filtered. The sulfurated potash solution is then added slowly to the zinc sulfate solution with constant stirring. Then the remainder of the water is added, and the lotion is mixed.

The subject matter claimed is:

1. A skin care composition comprising an admixture of a base and a xanthone-containing plant extract.

2. The skin care composition of claim 1 comprising about 50 to about 99.999 parts by weight of the base and about 1×10⁻³ to about 50 parts by weight of the xanthone-containing plant extract.

3. The skin care composition of claim 1 wherein the base comprises an oleaginous base.

4. The skin care composition of claim 3 wherein the oleaginous base comprises a hydrocarbon base.

5. The skin care composition of claim 3 wherein the oleaginous base comprises a silicone polymer.

6. The skin care composition of claim 3 wherein the oleaginous base comprises a vegetable oil.

7. The skin care composition of claim 3 wherein the oleaginous base comprises an animal fat.

8. The skin care composition of claim 1 wherein the base comprises an absorption base.

9. The skin care composition of claim 1 wherein the base comprises an emulsion base.

10. The skin care composition of claim 9 wherein the base comprises an aqueous phase, an emulsifying agent, and an oleaginous phase.

11. The skin care composition of claim 1 wherein the base comprises a water-soluble base.

12. The skin care composition of claim 11 wherein the water-soluble base comprises a member selected from the group consisting of polyethylene glycols, bentonite, colloidal magnesium aluminum silicate, sodium alginate, glyceryl monostearate, cellulose derivatives, and mixtures thereof.

13. The skin care composition of claim 11 wherein the water-soluble base is a cellulose derivative selected from the group consisting of methylcellulose, hydroxyethyl cellulose, and sodium carboxymethyl cellulose, and mixtures thereof.

14. The skin care composition of claim 1 further comprising a member selected from the group consisting of proteins, vitamins, botanical extracts, lipids, glycolipids, polymers, copolymers, and mixtures thereof.

15. The skin care composition of claim 1 further comprising a member selected from the group consisting of fragrances, coloring agents, emulsifiers, wetting agents, dispersing agents, penetrants, emollients, detergents, hardeners, preservatives, and mixtures thereof.

16. A method for making a skin care composition, the method comprising admixing a base and a xanthone-containing plant extract.

17. The method of claim 17 wherein the skin care composition comprises about 50 to about 99.999 parts by weight of the base and about 1×10⁻³ to about 50 parts by weight of the xanthone-containing plant extract.

18. A method for caring for skin, the method comprising applying to the skin a composition comprising an admixture of a base and a xanthone-containing plant extract.

19. The method of claim 18 wherein the composition comprises about 50 to about 99.999 parts by weight of a base and about 1×10⁻³ to about 50 parts by weight of the xanthone-containing plant extract.
